(19) United States
(12) Patent Application Publication
(10) Pub. No.: US 2017/0136260 A1
(43) Pub. Date: May 18, 2017

(54) BEVERAGE MADE UP OF THERAPEUTIC AGENTS IN COMBINATION WITH A TRANSPARENT CAN THAT CAN BE LIT UP

(71) Applicant: Eric Campos, Fullerton, CA (US)

(72) Inventor: Eric Campos, Fullerton, CA (US)

(21) Appl. No.: 15/353,533

(22) Filed: Nov. 16, 2016

Related U.S. Application Data
(60) Provisional application No. 62/386,075, filed on Nov. 17, 2015.

(51) Int. Cl. A61N 5/06; F21L 4/00; F21K 2/06; A23L 2/52; A61K 36/185; A61K 45/06; A61K 35/66; A61K 9/00; F21V 3/00; F21V 7/00

(52) U.S. Cl. CPC A61N 5/0618; F21V 3/00; F21L 4/00; F21K 2/06; F21V 7/00; A61K 36/185; A61K 45/06; A61K 35/66; A61K 9/0095; A23L 2/52; F21Y 2115/10

(57) ABSTRACT

The present invention relates to an herbal extract beverage that in combination with a clear LED lit container will affect the individual's mood depending on the color of light used, the herbal extract compositions used and the wavelength and wavelength frequency of the light used.

### CROSS- REFERENCE TO RELATED APPLICATIONS

The present Utility patent application claims priority benefit of the U.S. provisional application for patent Ser. No. 62/386,075 entitled “A BEVERAGE MADE UP OF THERAPEUTIC AGENTS IN COMBINATION WITH A TRANSPARENT CAN THAT CAN BE LIT UP,” filed on 17 Nov. 2016, under 35 U.S.C. 119(e). The contents of this related provisional application are incorporated herein by reference for all purposes to the extent that such subject matter is not inconsistent herewith or limiting hereof.

### BACKGROUND

Field of the Invention

The present invention relates to a beverage made up of different herbal therapeutic agents to reach a desired mood or effect in combination with a clear can that lights up to a certain color and wavelength frequency that when used together will elicit a certain mood or feeling.

Description of the Related Art

Research shows that you can change your mood by the colors you surround yourself with. Scientists have studied the effect of color on our mood and way of thinking for many years. The effect of colors and emotion is currently a topic of much interest. Magazines such as Self, Cosmopolitan, McCall's, American Health and Psychology Today have articles describing the association between color and mood and about the way color affects how people feel.

Extensive psychophysiological research allows us to specify the arousing, relaxing, awareness sharpening, or pacifying effects of various colors. Such research originates in the early work of pioneers such as Jacob Liberman (Light: Medicine of the Future, 1984). For example, it is well established that so called cool colors (in the green-blue spectrum) have a tendency to stimulate the parasympathetic portion of the autonomic nervous system (ANS), leading to reduced pulse rate and relaxation. Conversely, so called warm colors (in the red-orange-yellow spectrum) have a tendency to stimulate the sympathetic portion of the ANS, leading to increased pulsed rate and arousal.

Color therapy is gaining acceptance in the scientific community. In England, a headteacher improved behavior in students using “Kaleidoscope Therapy” in which colored lights are projected around a therapy room, and children explore emotions through color.

Among the principle color hues (red, yellow, green, blue and purple) and five intermediate hues (yellow-red, green-yellow, blue-green, blue-purple and red-purple), the responses have been observed and documented.

Blue elicited a high number of positive emotional responses including feelings of relaxation, calmness, peace and hope. The color blue is associated with the ocean, beach, water or the sky and thus inducing relaxing and calming effects.

Green elicited positive emotional responses including calmness, happiness, comfort and peace. Green is associated with nature, grass, trees, and reminds someone of outdoors and springtime.

Yellow-Red was associated with the color of Autumn and Halloween. People felt happy and energized.

Red reminds people of being in love. The color red evokes emotions such as passion and romance.

Yellow reminds people of happiness and sunshine.

In the United States, pink has been used in jail cells to calm prisoners. Dr. Alexander Schauss Ph.D., director of the American Institute for Biosocial Research in Washington stated, (pink is a) tranquilizing color that saps your energy.

Color Changing LED lighting technology's advantages in efficiency and long-durability have been complimented by scientifically proven benefits of increased well-being, mood and significant health benefits.

A recent study published in PUBMED.gov was done on the relationship of light, light color and light frequency and its effects on the Central Nervous System:

The Impact of Modulated, Colored Light on the Autonomic Nervous System.

Ross K J, Guthrie F, Dumont J C.

Abstract

Context:

Scientists are now finding that light acts on individuals through multiple pathways, most notably the optic nerve that links to the brain's visual cortex, providing a pathway for the visual effects of light. The optic nerve also links to the more recently discovered retinohypothalamic tract, providing a pathway for the nonvisual effects of light.

Objective:

The purpose of this study was to evaluate the impact of modulated-light projections, perceived through the eyes, on the autonomic nervous system (ANS).

Design:

The research team designed a randomized, controlled, partially blinded study with three intervention groups and one control group.

Setting:

The study took place in two locations: (1) Midwestern State University, Wichita Falls, Tex., USA (40 participants) and Centre de Santé Satori, Québec, Canada (77 participants).

Participants:

The research team recruited 117 individuals, 89 women and 28 men, to participate in the study. Participants were normal healthy individuals who were 19 to 72 y old (average age=43 y).

Intervention:

Three types of light projections, each containing both specific colors and specific modulations in the frequency range of brainwaves, were tested, in addition to a placebo projection consisting of nonmodulated white light.

Outcome Measures:

Evaluation was done using a combination of physiological measures-heart rate (HR), heart rate variability (HRV), and skin conductance (SC)- and psychological tests: the Profile of Mood States (POMS) and a subjective evaluation questionnaire.

Results:

The research team observed significant differences in the effects of light-modulation projections from baseline to postsession as compared with an equivalent intensity of white light, including decreased HR, increased HRV standard deviations of normalized NN (beat-to-beat) intervals (SDNN), very low (VLF) and low frequency (LF) levels, and decreased POMS total mood disturbance (TMD). Also, the different colors of modulated light were found to result in different ANS effects.

Conclusions:

Interest is growing in the therapeutic potential of light. The effects demonstrated in the current study indicate that colored light could significantly enrich the therapeutic potential of light, and further research into chromotherapy is warranted.

PMID: 24067320

[PubMed—indexed for MEDLINE]

Physiological responses to stressful conditions are generally the same; only the intensity of the response and whether or not any given response will be evoked are individual in nature. Acute stressful situations, such as resulting from a trauma, produce a physiological response, after which the body returns to its normal, un-stressful state. When the body enters a stressful situation, the sympathetic nervous system invokes a fight or flight response. Once the stressful stimuli have been removed, the parasympathetic nervous system returns the body to a normal state.

Chronic stressful conditions, caused by for example a divorce, an unpleasant boss, lack of money, or building a new home, are more insidious. The physiological response endures and the body fails to return to the baseline state. Being in a continuous state of stress, an individual will feel unwell, partly because the mechanisms that ordinarily help overcome

There is an ever-increasing interest in herbal or natural-source remedies or medications. Many individuals would rather use such products than conventional pharmaceutical preparations. Additionally, medicinal substances derived from natural products can provide commercial or industrial opportunities for local populations in areas where medicinal plants grow or are cultivated. Moreover, compounds identified as the active ingredients in natural products form an important basis for pharmaceutical research.

Herbal extracts and vitamins can be used to alleviate pain, reduce stress, stimulate or relax the body.

While herbal extract drinks, light modulation fixtures and color changing mood lights are being used individually to treat mood disorders, a “mood drink” that combines all 3 modalities is currently unavailable. There is no coordination between the colors of the light emitted and the herbal composition of the liquid drink.

Thus, there is a need for a therapeutic beverage combined with a colored LED light source used to alleviate or at least reduce the stress and symptoms associated therewith. Furthermore, there is a need for a mood enhancing beverage/light combination that uses the color of the light, the correct frequency of light and the correct composition of ingredients of the liquid to achieve a desired positive emotional response including: calmness, happiness, energy, passion, focus and creativity.

### SUMMARY OF THE INVENTION

It is, therefore, one of the principal objects of the present invention to provide an herbal extract beverage that in combination with a clear LED lit container will affect the individual's mood depending on the color of light and frequency used and the herbal extract composition used.

In an embodiment, the combination of the clear can used with a “blue” colored LED light and a liquid composition that has calming herbs and vitamins, can be marketed as a “calming” drink.

In another embodiment, the combination of the clear can used with a “yellow” colored LED light and a liquid that has stress reducing extracts and vitamins, can be marketed as a “happiness or joy” drink.

In another embodiment, the combination of the clear can used with a “red” colored LED light and a liquid that has sexual wellness inducing extracts, can be marketed as a “passion or love” drink.

In another embodiment, the combination of the clear can used with an “orange” colored LED light and a liquid that has energy enhancing extracts and vitamins, can be marketed as an “energy and creativity” drink.

In another embodiment, the combination of the clear can used with a “lime” colored

LED light and a liquid that has mental focus and clarity extracts and vitamins, can be marketed as a “focus” drink.

In another embodiment, the combination of the clear can used with an “amber” colored LED light that also filters out the blue light spectrum and a liquid that has sleep extracts and vitamins, can be marketed as a “sleep” drink.

A further object of the present invention is to provide a short lasting, low cost, battery powered LED light source that is used at the base or lid of clear container in order to achieve the desired color of light and the correct frequency of the light wavelength.

### BRIEF DESCRIPTION OF THE DRAWINGS

The present invention will become more fully understood from the detailed description and accompanying drawings, which are not to scale, wherein:

FIG. 1. is a cross-sectional view of a clear PET can combination with LED light source in accordance with the teachings of the disclosure;

FIG. 2 is an exploded view of the clear PET can and LED light source of FIG. 1

FIG. 3 is a perspective top view of a preferred embodiment with the removable “pop-off' can top in accordance with the teachings of the disclosure;

FIG. 4 is a perspective side view of the clear PET can with a mirrored reflector showing on the inside of can in accordance with the teachings of the disclosure.

While the present disclosure is susceptible to various modifications and alternative constructions, certain illustrative embodiments thereof have been shown in the drawings and will be described below in detail. It should be understood, however, that there is no intention to limit the present invention to the specific forms disclosed, but on the contrary, the intention is to cover all modifications, alternative constructions, and equivalents falling with the spirit and scope of the present invention.

### DETAILED DESCRIPTION

The following detailed description of the preferred embodiment is an example and is not intended to limit the invention, its application or its uses.

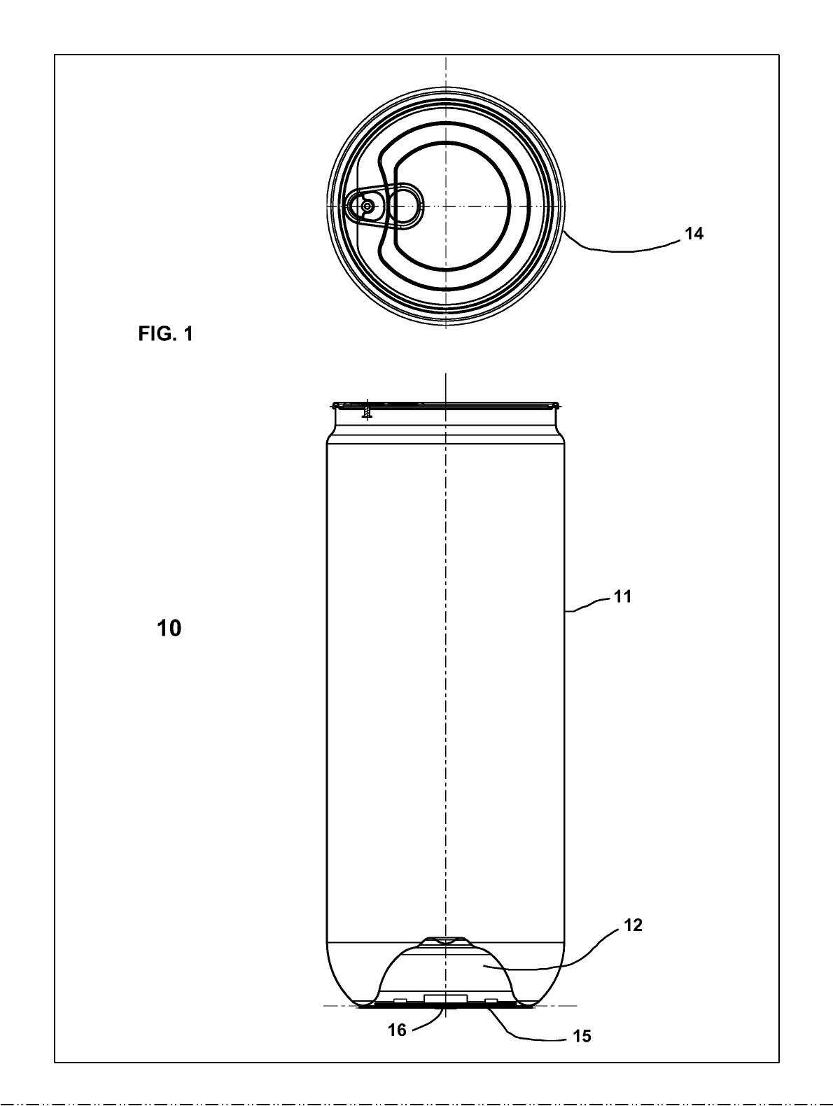

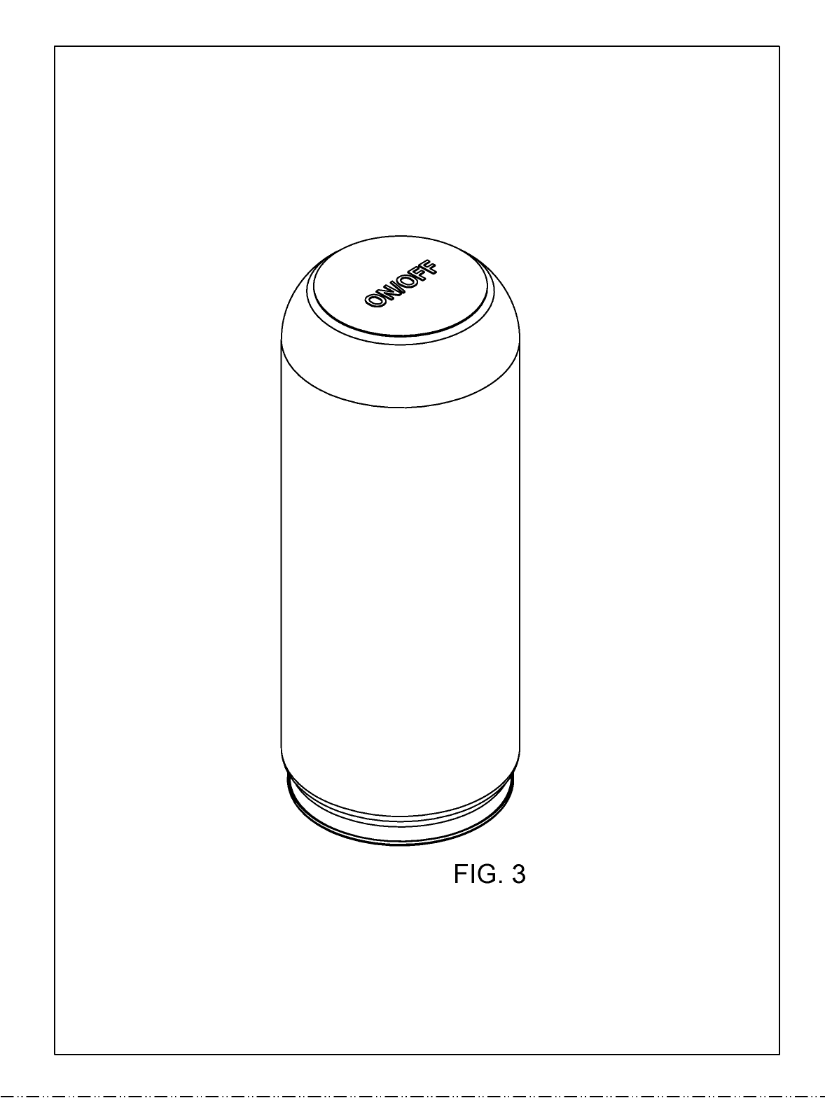

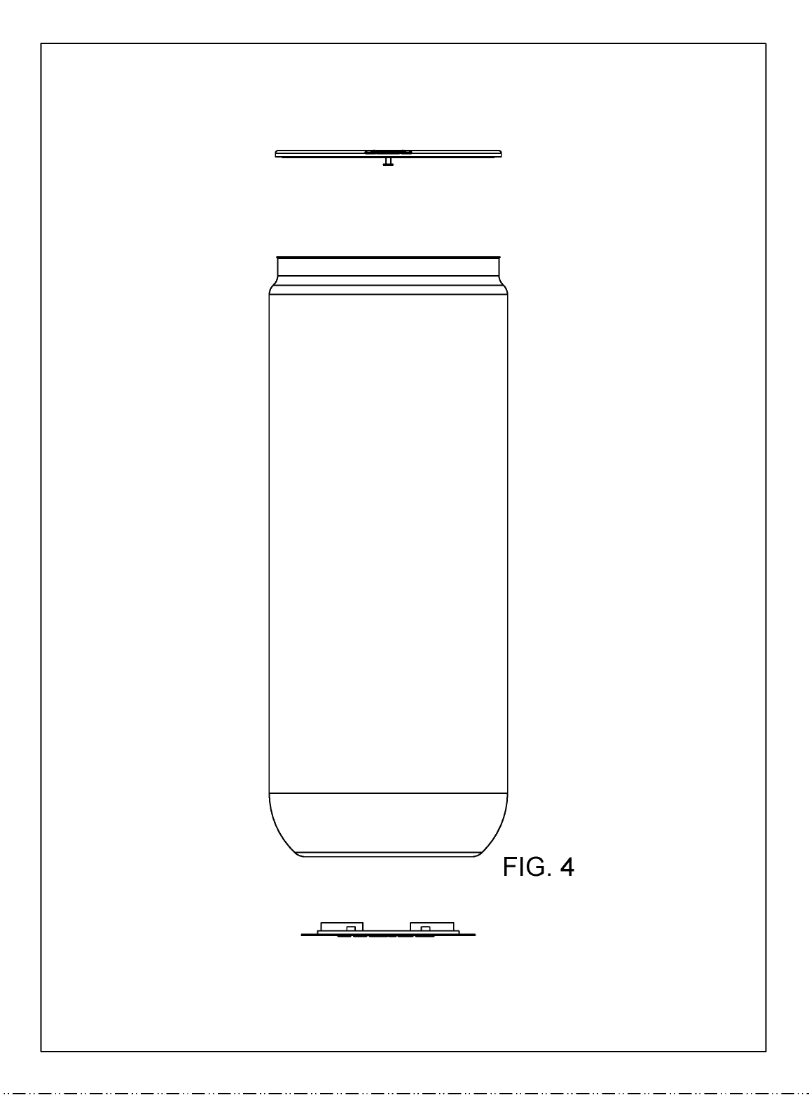

Referring to the figures, FIG. 1 is a side cross-sectional view of a clear can combination with LED light source and will be referred to as reference numeral 10. With further reference to FIGS. 3 and 4, clear PET can with removable top and mirrored reflector will be referred to as reference numeral 30. It is understood that the teachings of the disclosure can be used to construct clear cans and lighting circuits above and beyond that specifically disclosed below. One of ordinary skill in the art will readily understand that the following are exemplary embodiments.

One example of a clear can device that may use LED lighting circuitry described herein is shown in FIG. 1. The single color or color changing LED light source circuit 15 coupled to the base of clear PET can 11 can be used to illuminate liquid inside the can. As described in U.S. Pat Nos. 7,476,002/8,480,248/8,827,496/6,847,909 and US Publication No. 20100283407 all of which are incorporated herein by reference, the combination PET can 11 and LED Light source 15 can be used to enhance the mood of the consumer and the surroundings. The clear PET can 11 is designed with a concave base 12 allowing for the LED light source circuit 15 to be mounted flush at base of can. The LED light circuit 15 has a low profile button switch 16 providing a means to change the color or wavelength frequency of the LED light source. The two disposable batteries 17 provide the LED light circuit with at least 3 hours of continuous light use. The lid 14 can be permanent or removable.

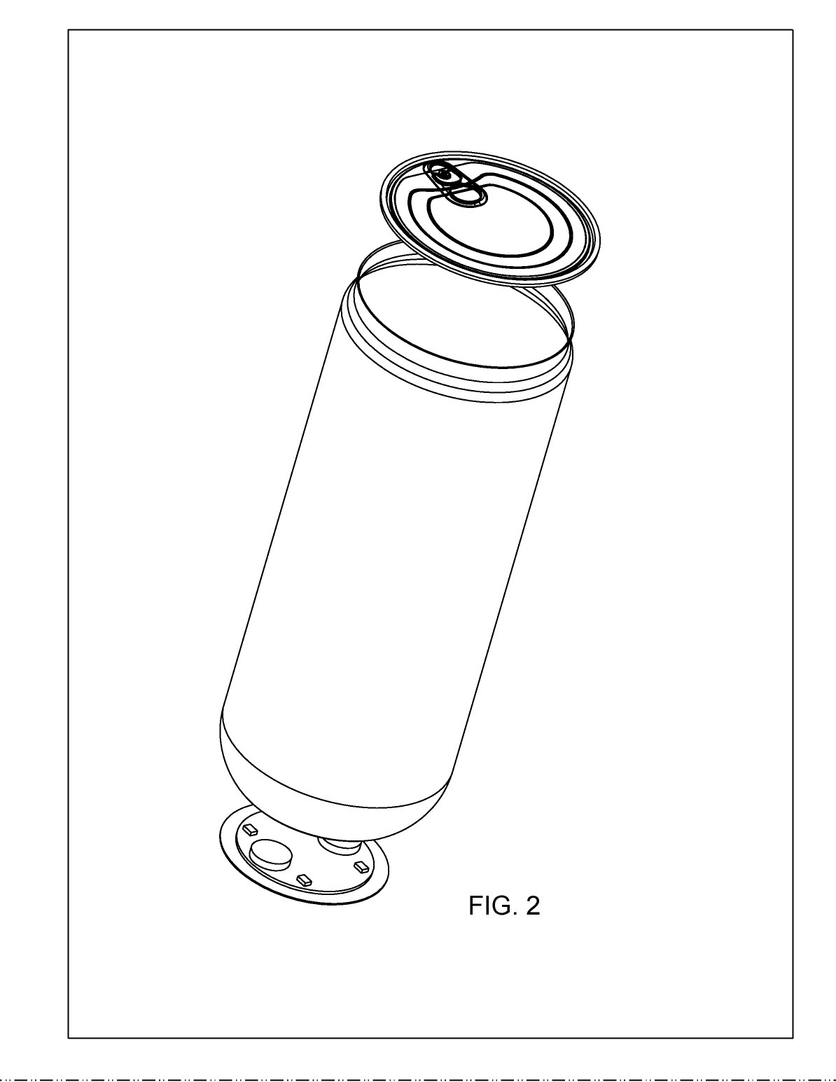

Turning to FIG. 2, an exploded view 10 of the clear PET can 11 with LED circuit 15 is shown. LED light circuit 15 is shown at base of clear PET can 11. Of course, the can size and shape of this disclosure is for illustration only and may be embodied with any size and shaped can. An aluminum top 14 is used to seal the can and serves as a reflector for the LED light source while also maintaining the look of a standard aluminum drink can.

Turning to FIG. 3, the versatility of the design of this disclosure is particularly illustrated. As shown in FIG. 3, the can device 30 with LED light circuit 15 may include a removable “pop-off” top 20. The pop-off top 20 can be used in a bar setting where the bartender can remove the top from the can, add ice and quickly serve to patrons without having to pour liquid contents into another glass. The possible configuration and function of the removable tops are described in U.S. Pat. Nos. 7,891,519 and D739,728 all of which are incorporated herein by reference.

Regarding the use of herbal liquid extracts, the disclosed can devices may incorporate a liquid composition and mixture of ingredients meant to enhance the mood of the consumer. The mood enhancing extracts can be made in any number of combinations. For example, the combinations as described in U.S. Pat. Nos. 8,263,667 and US Publication Nos. 20140343134, 201502833190, 20150272989 or 20150174074 all of which are incorporated herein by reference.

Turning to FIG. 4, the device 30 with LED light circuit 15 may incorporate a reflective mirror 22 on the interior wall of clear can 11. The reflector serves as a focusing point for the modulated light making it easier for the visual cortex to pick up the light waves. The LED circuit 15 can use single color LEDs 18 or RGB LEDs operated in any number of combinations to provide specific color of light or color patterns as described in U.S. Pat. No. 8,579,795. It also serves as a light wave reflector that projects different lighting patterns into the surrounding area.

Thus it can be seen that a beverage with the correct liquid compositions when used with an illuminated clear container that modulates the frequency and color of the light improves upon prior art. Although the preceding description contains significant detail in relation to the preferred embodiments, it should not be construed as limiting the scope of the invention but rather as providing illustrations of the preferred embodiments.

1. An herbal and vitamin liquid beverage that when used in combination with a clear LED lit container will affect the individual's mood depending on the color, wavelength and frequency of that wavelength used and the herbal and vitamin liquid compositions used. Wherein the LED circuit is designed to modulate the frequency of the light to elicit a certain physical and emotional response ranging from calm to alertness.

2. The beverage and lit container combination according to claim 1, wherein the liquid composition has Ultra Violet (UV) reactive properties and is used in combination with a 385 nm-395 nm wavelength UV LED light for the purpose of creating a neon glow effect on the liquid composition.

3. The beverage and lit container combination according to claim 1, wherein the liquid composition is made up of “calming” herbs, vitamins and minerals and is used in combination with a blue LED light or a blue color shade thereof for the purpose of inducing calmness.

4. The beverage and lit container combination according to claim 1, wherein the liquid composition is made up of “energizing” herbs, vitamins and is used in combination with an orange LED light or an orange color shade thereof for the purpose of inducing an energizing and creativity effect.

5. The beverage and lit container combination according to claim 1, wherein the liquid composition is made up of “sexually enhancing” herbs, vitamins and minerals and is used in combination with a red LED light or a red color shade thereof for the purpose of inducing a sexually enhancing and increased blood flow effect.

6. The beverage and lit container combination according to claim 1, wherein the liquid composition is made up of “mental focus enhancing” herbs, vitamins and minerals and is used in combination with a lime (yellow-green) LED light or a lime color shade thereof for the purpose of inducing a state of mental focus and clarity.

7. The beverage and lit container combination according to claim 1, wherein the liquid composition is made up of “happiness and positive outlook enhancing” herbs, vitamins and minerals and is used in combination with a yellow LED light or a yellow color shade thereof for the purpose of inducing a happiness and joyful effect.

8. The beverage and lit container combination according to claim 1, wherein the liquid composition is made up of “sleep enhancing” herbs, vitamins and minerals and is used in combination with an amber LED light with a blue light filter to help enhance the production of melatonin for the purpose of helping induce sleep.

9. An herbal and vitamin liquid beverage that when used in combination with a clear LED lit container will affect the individual's mood depending on the color of light used, the herbal and vitamin liquid compositions used and the wavelength and wavelength frequency of the light used. Wherein said LED light source is a disposable battery powered RGB color changing LED light circuit that can be used for at least several hours.

10. An herbal and vitamin liquid beverage that when used in combination according to claim 9 with a clear LED lit container will affect the individual's mood depending on the color of light used and the herbal and vitamin liquid compositions used. Wherein said light source is a non-battery chemical based neon light puck similar to the neon “Night Sticks”.

11. An herbal and vitamin liquid beverage that when used in combination according to claim 9 with a clear LED lit container will affect the individual's mood depending on the color of light used, the herbal and vitamin liquid compositions used and the wavelength and wavelength frequency of the light used. Wherein the clear container is a transparent PET can with a “Pop-Off” top allowing the drink to be used as a cocktail mixer by just adding ice and any flavor desired.

12. An herbal and vitamin liquid beverage that when used in combination according to claim 9 with a clear LED lit container will affect the individual's mood depending on the color of light used, the herbal and vitamin liquid compositions used and the wavelength and wavelength frequency of the light used. Wherein the clear container is a transparent PET can with a custom lid that holds all the dry ingredients including, color, vitamins and minerals in a membrane pocket allowing the drinker to release the ingredients just before consuming.

13. An herbal and vitamin liquid beverage that when used in combination according to claim 9 with a clear LED lit container will affect the individual's mood depending on the color of light used, the herbal and vitamin liquid compositions used and the wavelength and wavelength frequency of the light used. Wherein the clear container is a transparent PET can with a mirrored reflector attached to inside of can allowing for the optic nerve of the eyes to better capture the frequency and wavelength of light for enhanced effect.

14. An herbal and vitamin liquid beverage that in combination according to claim 9 with a clear LED lit container will affect the individual's mood depending on the color of light used and the herbal and vitamin liquid compositions used and the wavelength and wavelength frequency of the light used. Wherein the clear container is a transparent glass bottle.

15. An herbal and vitamin liquid beverage that in combination according to claim 9 with a clear LED lit container will affect the individual's mood depending on the color of light used and the herbal and vitamin liquid compositions used. Wherein a single use blister of aroma oils is attached to clear can adding an aromatic therapy experience.

16. An herbal and vitamin liquid beverage that when used in combination according to claim 9 with a clear LED lit container will affect the individual's mood depending on the color of light used and the herbal and vitamin liquid compositions used without the use of light modulation.

17. An herbal and vitamin liquid beverage that when used in combination according to claim 9 with a clear LED lit container will affect the individual's mood depending on the color of light used and the herbal compositions used wherein said herbal composition is made up of Distilled Cannabis Extracts or Concentrates.

18. A vitamin liquid beverage that when used in combination according to claim 9 with a clear LED lit container will affect the individual's mood depending on the color of light used and the liquid compositions used wherein said liquid composition is made up of probiotic and prebiotic strains.
